(19) United States
(12) Patent Application Publication
(10) Pub. No.: US 2007/0041931 A1
(43) Pub. Date: Feb. 22, 2007

(54) Compositions Comprising Soy Products and Dioic Acids

(76) Inventor: Muriel Morelli, Acquigny (FR)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/421,097

(22) Filed: May 31, 2006

(30) Foreign Application Priority Data
Oct. 27, 2004 (WO) PCT/EP04/12163
Oct. 29, 2003 (EP) 03292705.5

(51) Int. Cl. A61K 8/97; A61K 31/19; A61K 36/48

(52) U.S. Cl. 424/74; 424/757; 514/574

(57) ABSTRACT

This invention relates to topical compositions comprising soy products and dioic acids and their use in skin lightening, against hyperpigmentation and, in particular, against skin spots.

### BRIEF DESCRIPTION OF THE INVENTION

This invention relates to topical compositions comprising soy products and dioic acids and their use in skin lightening, against hyperpigmentation and, in particular, against skin spots.

### BACKGROUND OF THE INVENTION

As a first sign of aging, skin becomes less elastic and develops fine lines and wrinkles, which result from the deterioration of the dermis layer. Indeed the skin's ability to replace damaged collagen diminishes and more caps and irregularities develop in the collagen network. This goes along with the appearance of pigment marks, skin thinning and skin sagging. Many factors contribute to skin aging, including sun exposure, free radicals, some age-related hormonal changes, and smoking.

A number of treatments have been developed that have proved out to be more or less effective in combating the effects of skin aging. These include applying cosmetic products which contain vitamins or vitamin derivatives, in particular vitamin A or its derivatives, alpha-hydroxy acids, retinoids, vitamin C, or plant extracts. Aging and exposure to sunlight are factors that contribute to the development of so-called “skin spots” or “age spots”. Changes in the epidermal cells, in particular in those which are pigment producing, i.e. the so-called melanocytes, may lead to hyperpigmentation.

A class of products that are used against the effects of sking aging but also have other beneficial effects on the skin are soy-derived products. Soy products and in particular non-denatured soy products are known to retard hair growth as is described in EP-A-1074240. The latter describes compositions and methods for delaying hair growth, reducing hair follicle and hair shaft size and hair shaft pigmentation by topically applying to the skin a composition comprising legume extracts including soymilk

WO 01/34099 describes the use of non-denatured soy product containing compositions for depigmentation, evening out skin tone and skin texture, skin firming and care of the skin. U.S. Pat. No. 6,555,143 discloses compositions and methods that relate to legume products and in particular to soy products for regulating firmness of the skin, hair or nails; cleansing the skin, hair or nails; reducing and/or delaying hair or nail growth; and a number of other useful applications. EP-A-1236465 describes legume products having trypsin inhibitory activity, in particular soy products, having reduced microbial content and the use thereof in compositions for application on the skin, nails and hair.

EP 662 818 (Unilever) describes combinations of retinol or derivatives thereof with certain dioic acids for the treatment of age spots.

Although existing products for improving the appearance of the skin and/or for combating the effects of skin aging have been applied with varying degrees of success, there nevertheless remains room for improvement. In particular there still is a need for new formulations that are more effective in combating the effects of skin aging and/or that improve the appearance of the skin and in particular of age spots. Providing compositions that possess some or several of these effects or properties is an object of this invention.

### SUMMARY OF THE INVENTION

The present invention is directed to a topical composition comprising a soy product and at least one dioic acid which can be represented by the formula HOOC-Alk-COOH, wherein Alk represents a bivalent hydrocarbon radical. The soy product in particular can be a non-denatured soy product, e.g. non-denatured soymilk or powder.

The topical composition can be for pharmaceutical or dermatological use but is in particular for cosmetic use.

The topical compositions in particular are useful for combating and treating hyperpigmentation of skin, in particular against age spots. They are also useful for increasing skin firmness and elasticity, even tone and texture, to combat and treat the effects of skin aging, to prevent and treat sun-induced damage and acne.

Or, alternatively, the invention concerns a method, and in particular a cosmetic method of combating or treating the effects of skin aging and/or improving the appearance of the skin, including increasing skin firmness and elasticity, evening tone and texture, preventing and treating sun-induced damage and acne, controlling or reducing skin pigmentation, which method or cosmetic method comprises applying to the affected skin area an amount of a composition as defined herein, said amount being effective to treat said effects of skin aging and/or improve the appearance of the skin.

Still another aspect of this invention comprises a cosmetic method for the improvement of the external appearance of an individual, said method comprising applying a composition or a topical composition as defined herein to affected skin areas.

### DETAILED DESCRIPTION OF THE INVENTION

The compositions of the present invention contain soy products that may be in the form of a fluid (e.g., soymilk) or a solid (e.g., a soybean powder or soymilk powder). What is meant by “soy product” is a substance derived from the soybean, containing the ingredients naturally found in soybeans, at the relative concentrations as found in the beans. In preferred embodiments, the soy product is a non-denatured soy product. The latter is a soy product, which has been obtained by processes that leave the active proteins intact by carefully controlling the process parameters such as the temperature, the extraction media. This can be measured, for example, by the presence of intact soybean trypsin inhibitor (STI) protein.

In another embodiment, the soy product is soymilk One way to make soymilk is to soak the soybeans in deionized or purified water for several hours, and grind them after they were fully hydrated, with the addition of small quantities of water. (The grinding process allows the soybean milk to be extracted). After collection, the soybean milk may be filtered to remove any residual parts of the bean husk. The soymilk used in this invention can be fresh soymilk as described above, or may be made from soybean powder and water. The soybean powder is milled from soybeans and may also be lyophilized, spray dried, or freeze-dried and the resulting soymilk may or may not be filtered. Such prepared soymilk may have from about 1 to about 90% by weight dry soybean powder. Another example is the use of soymilk powder, made from lyophilized, spray dried or freeze-dried soymilk, with the addition of water and finished with or without filtration or homogenization.

Other methods of soybean extraction could also be used to create the active ingredients used in this invention. For example, but not limited to, the active ingredients could be extracted from ground soybeans using ethanol/water mixtures, followed by the removal of the ethanol from the extract, in such ways that the protease inhibitory activity of the soybean will be retained.

The soy products useful in this invention may be produced from all soybean species, regardless of their geographic origin, sun exposure, harvest time and the like. However, specific strains, geographic origins or growth conditions might be preferred.

For example, but not limiting to, soybean strains particularly rich in their trypsin inhibitor (e.g. STI, LTI, BBI) content or growth conditions that result in trypsin inhibitor enrichment in the bean, may be preferred. It should be noted that the soy products useful in the compositions of this invention may have a distinctive odor, which may be tolerable in some cultures, but is undesired in others. If necessary, the odor of the compositions of this invention may be reduced by using soybean products derived from specific strains of soybeans known to produce reduced-odor, including, but not limited to, lipoxygenase-2-deficient beans and those having modified sugar profile, and the like. A process to reduce oxygen levels in the formulation may also reduce the odor. Various masking agents or fragrances may also be used to mask the odor.

Of particular interest are soy products derived from soy strains that are rich in sucrose, in particular soy milk of high sucrose content and any soy product derived therefrom Preferred for use in the compositions of the present invention are non-denatured soy products, in particular non-denatured soy products that are rich in sucrose. These are preferably decontaminated as described in EP-1236465, for example by gamma irradiation of non-denatured soymilk powder, preferably at a dose of about 10 kGy.

The compositions of the invention further contain at least one dioic acid of formula HOOC-Alk-COOH as specified above. The radical Alk is a bivalent hydrocarbon radical. The radical Alk may have from 8 to 40 carbon atoms, in particular from 8 to 30 carbon atoms, more in particular from 8 to 22 carbon atoms, still more in particular from 8 to 18 carbon atoms. Said radical may be saturated or unsaturated, either mono- or polyunsaturated, e.g. mono- or di-unsaturated. The dioic acid may occur in isomeric forms, e.g. as cis or trans isomers or cis/trans isomeric mixtures. Of particular interest is octadecenedioic acid or octadecadienedioic acid or mixtures thereof, in particular a octadecenedioic acid mixture which is commercially available under the tradename Arlatone™ Dioic DCA.

C₈₋₁₈ saturated dioic acids are commercially available. Other dioic acids can be made by art-know methodologies. Unsaturated dioic acids can be made by enzymatic procedures, e.g. starting from unsaturated esters, preferably in the form of glyceride esters as described in EP 662 818.

In a preferred embodiment, there is provided a composition comprising at least one soy product as defined herein and a C₁₈ dioic acid, in particular a mono- or di-unsaturated C₁₈ dioic acid, including a cis/trans isomeric form thereof and further including mixtures thereof.

The compositions of this invention may contain a stabilizing system. The latter may for example comprise one or more components selected from the group consisting of one or more antioxidants, chelating agents and preservatives, as described in WO 01/34099, which is incorporated herein by reference.

Thickening agents (e.g., thickeners or viscosity enhancing agents) may be utilized in the compositions of this invention to alter their viscosity. The desired viscosity of the composition will depend upon the intended use (e.g., as a bath product, cream, lotion, or gel). For example, in applications such as bath or wash products, the viscosity of the composition should be relatively low, similar to an aqueous solution. Application as a cream, lotion, or gel will have slightly higher viscosity (e.g., between about 100 cPs and 100,000 cPs).

Thickening agents that can be added to the compositions of this invention to alter viscosity include polymers such as polyacrylates (e.g., polyacrylamide). Other examples of viscosity modifying agents are listed on pages 1692-97 of the Cosmetic Handbook. To achieve the appropriate viscosity, compositions of the present invention may comprise from about 0.01% to about 20%, by weight (e.g., from about 0.1% to about 5%, by weight) of a thickening agent.

The compositions of the invention are prepared by adding the dioic acid to the soy component or vice versa.

In the compositions of the present invention the w/w ratio of the soy product to the total amount of the said dioic acids may vary but in particular is in the range of about 50:1 to 1:1, further in particular from 30:1 to 1:1, still further in particular from 20:1 to 2:1, preferably from about 10:1 to 2:1, more preferably the w/w ratio is in the range of about 10:1 to 3:1. These w/w ratios relate to the total amount of dry soy product and dry dioic acids.

The topical compositions of this invention comprise as well dermatological formulations (or topical pharmaceutical formulations), and cosmetical formulations. Said topical compositions may further contain other ingredients or additives used in dermatological or in cosmetic formulations, including other active ingredients. Examples of further ingredients or additives are surfactants, emulsifiers, consistency factors, conditioners, emollients, skin caring ingredients, moisturizers, thickeners, lubricants, fillers, binding agents, anti-oxidants, preservatives, active ingredients, in particular dermatologically active ingredients, fragrances and the like. Active ingredients as mentioned herein comprise, for example, anti-inflammatories, anti-bacterials, anti-fungals and the like agents. Of particular interest are any active ingredients suited for topical applications.

The topical formulations according to the invention can further include one or more of a variety of optional ingredients, such as coloring agents, opacifying agents and the like.

The soy product preferably is used in the topical compositions at concentrations from 0.001% to 10% and preferably from 0.1% to 5%, more preferably from 1% to5%. These percentages refer to solid soy product, e.g. soybean powder or soymilk powder. Compositions, which comprise solid soy products may also comprise water (e.g., distilled water or water contained within soymilk) to form a liquid base to the composition (e.g., to form a cream, lotion or gel). Such composition may comprise from about 50% to about 98% by weight (e.g., from about 70% to about 98%, by weight) of water. A liquid soy product (e.g., soymilk) may also be used, in that instance the quantities of liquid soy product need to be calculated in function of the quantity of water present in the liquid soy product. The dioic acids taken together should be used in the formulations for topical application between about 0.01% to 5%, preferably from about 0.01 to 2%, more preferably from about 0.1% to 1%.

Unless indicated otherwise, all percentages in the preceding and following paragraphs are w/w percentages.

The formulations according to the present invention can be prepared by adding the appropriate ingredients to a composition of the invention, or vice ve rsa by adding the composition to an appropriate cosmetic or dermatological formulation base. It is also possible to mix all the ingredients individually, i.e. without making a separate composition as defined herein.

The compositions according to the present invention can be prepared by mixing the appropriate ingredients. It is also possible to make premixes and to add ingredients or other premixes. In a preferred method of preparation, the compositions of this invention are emulsion-based, in particular oil-in-water emulsions and are made by preparing an aqueous phase containing all hydrophilic components, an oil phase containing all lipophilic components, and subsequently adding the oil phase to the aqueous phase as to prepare an emulsion. Preferably, a premix of the soy product in some water is added after the formation of the emulsion.

The compositions subject of the present invention are useful to combat or to treat the effects of skin aging. The effects of skin aging comprise those associated with the aging of the skin such as the appearance of fine lines, fine wrinkling, wrinkling, loss of skin firmness, skin tightening and suppleness. The effects of skin aging are due to aging as such, but also to aging of the skin caused by external factors such as exposure to environmental factors such as exposure to sunlight, wind, atmospheric poluants and the like, or a combination of these factors.

Soy products and in particular any non-denatured soy products as mentioned herein, are known to have a number of beneficial effects such as increasing skin firmness and elasticity, even tone and texture to inhibit skin pigment formation, to combat and treat the effects of skin aging and to stimulate the proliferation of dermal cells and in particular the proliferation of fibroblasts, to increase glycoaminoglycan and stimulate collagen formation, to prevent and treat sun-induced damage and acne. Additionally, they have been found to be effective as hair growth retardants. These effects and properties are even more present when the soy products are non-denatured soy products.

It now has been found that in the composition of this invention, the soy products mentioned in this specification and claims and the dioic acids specified herein, act synergistically on a number of the beneficial properties of these components, in particular in terms of their action on skin pigmenting and more in particular on age spots.

The topical compositions according to the invention may be in the form of a solution, a hydrophilic lotion, an ointment, a cream, a gel or a spray. The formulations may also be, for example, in the form of oil-in-water, water-in-oil or multiple emulsions, foaming products or in liposome form.

Preferred formulations are gel and cream based.

All the topical formulations as described above can be applied on the skin by means of wipes.

The topical formulations of the invention may be applied in the morning and/or evening. They may be applied on those parts of the body where skin aging is prominent, i.e. on the face, the body or the hands.

The following examples are meant to illustrate the invention and not to limit it thereto.

### EXAMPLE

In the following formulation example all percentages are by weight (w/w).

The formulations are prepared in the following manner. First an aqueous phase is made which is heated. Subsequently the gellyfying agents are added.

The oily components are melted and mixed into an oily phase, which is emulsified in the water phase. The thus formed emulsion is allowed to cool and the active ingredients are added at a temperature of 35° C. Then the fragrance is added at a temperature of 30° C.

High sucrose soy milk powder is an ingredient obtained from soy milk with high sucrose content as described in EP-1236465.

| INCI Name | % (w/w) |
|---|---|
| Aqua | 69.53 |
| Disodium EDTA | 0.20 |
| Glycerin | 4.00 |
| C12-15 Alkyl Benzoate | 2.50 |
| Arachidyl Alcohol | 1.50 |
| Behenyl Alcohol | |
| Arachidyl Glucoside | |
| Cetearyl Alcohol | 3.00 |
| Cetearyl Glucoside | |
| BHT | 0.07 |
| Ethylhexyl Methoxycinnamate | 6.50 |
| Butyl Methoxydibenzoylmethane | 3.00 |
| Propylparaben | 0.20 |
| Methylparaben | 0.35 |
| Octadecenedioic Acid | 1.00 |
| Polyacrylamide | 2.00 |
| C13-14 Isoparaffin | |
| Laureth-7 | |
| Aqua | |
| High Sucrose Milk Powder | 2.00 |
| Phenoxyethanol | 0.80 |
| Chlorhexidine Digluconate | 0.25 |
| Dimethicone | 1.00 |
| Aluminum Octenylsuccinate | 2.00 |
| PEG-4 | 0.10 |
| PEG-4 Laurate | |
| PEG-4 Dilaurate | |
| Iodopropynyl Butylcarbamate | |
| Total | 100.00 |

1. A composition comprising at least one soy product and at least one dioic acid, which can be represented by the formula HOOC-Alk-COOH, wherein Alk represents a bivalent hydrocarbon radical.

2. A composition according to claim 1 wherein Alk in the dioic is a bivalent hydrocarbon radical having from 8 to 40 carbon atoms, in particular from 8 to 30 carbon atoms, more in particular from 8 to 22 carbon atoms.

3. A composition according to claim 2 wherein the dioic acid is a C₁₈ dioic acid.

4. A composition according to any of claims 1-3, wherein the soy product is a non-denatured soy product.

5. A composition according to any of claims 1-4, wherein the w/w ratio of the soy product to the total amount of the said dioic acids may vary but in particular is in the range of about 50:1 to 1:1, further in particular from 30:1 to 1:1, still further in particular from 20:1 to 2:1, preferably from about 10:1 to 2:1, more preferably the w/w ratio is in the range of about 10:1 to 3:1 and wherein the w/w ratios relate to the total amount of dry soy product and dry salts.

6. A composition according to claims 1-5, wherein the soy product is used at concentrations from 0.001% to 10% and preferably from 0.1% to 5%, more preferably from 1% to5%.

7. A formulation according to claim 1-5 wherein the dioic acids taken together are present at concentrations from about about 0.01% to 5% and preferably from about 0.1 to 2%, more preferably from about 0.1% to 1%.

8. Use of a composition as claimed in claims 1-5 for increasing skin firmness and elasticity, even tone and texture, to combat and treat the effects of skin aging, to prevent and treat sun-induced damage and acne.
